(19) United States
(12) Patent Application Publication
(10) Pub. No.: US 2024/0325276 A1
(43) Pub. Date: Oct. 3, 2024

(54) Personal Care Compositions

(71) Applicant: Colgate-Palmolive Company, New York, NY (US)

(72) Inventors: Alexandra D'ARCANGELIS, Randolph, NJ (US); Qiang WU, Hillsborough, NJ (US); Thomas BOYD, Metuchen, NJ (US); Erick CARDENAS ALPIZAR, Mexico City (MX); Aaron COHEN, New Brunswick, NJ (US); Belizze ZAGO, Mexico City (MX); Zhaoyu WEN, Guangzhou (CN); Yuyan ZENG, Guangzhou (CN); Dongyao HUANG, Guangzhou (CN)

(73) Assignee: Colgate-Palmolive Company, New York, NY (US)

(21) Appl. No.: 18/620,171

(22) Filed: Mar. 28, 2024

Related U.S. Application Data
(60) Provisional application No. 63/493,475, filed on Mar. 31, 2023.

(51) Int. Cl. A61K 8/67; A61Q 19/10

(52) U.S. Cl. CPC A61K 8/675; A61Q 19/10; A61K 8/676; A61K 8/678; A61K 2800/5922

(57) ABSTRACT

Personal care compositions and personal care compositions formulated to preserve pigment associated with a skin tattoo, improve the health skin, and/or improve the appearance of skin arc disclosed herein.

### CROSS-REFERENCE TO RELATED APPLICATIONS

This application claims the benefit of priority from U.S. Patent Application No. 63/493,475, filed Mar. 31, 2023, entitled Personal Care Compositions, the content of which is hereby incorporated herein in its entirety, for all purposes.

### BACKGROUND

A tattoo is a body modification made by inserting ink into the layers of skin to change the pigment for decorative or other reasons. The tattooing process involves the placement of pigment into the skin's dermis, the layer of connective tissue underlying the epidermis. After initial injection, pigment is dispersed throughout a homogenized damaged layer down through the epidermis and upper dermis, in both of which the presence of foreign material activates the immune system's phagocytes to engulf the pigment particles. As healing proceeds, the damaged epidermis flakes away (eliminating surface pigment) while granulation tissue forms deeper in the skin, which is later converted to connective tissue composed of extracellular matrix. This mends the upper dermis, where pigment remains trapped within fibroblasts, ultimately concentrating in a layer just below the dermis/epidermis boundary.

While the pigment remains trapped within the fibroblasts, the pigment may degrade over time due to exposure to sunlight and certain chemical compounds.

### BRIEF SUMMARY

This summary is intended merely to introduce a simplified summary of some aspects of one or more implementations of the present disclosure. Further areas of applicability of the present disclosure will become apparent from the detailed description provided hereinafter. This summary is not an extensive overview, nor is it intended to identify key or critical elements of the present teachings, nor to delineate the scope of the disclosure. Rather, its purpose is merely to present one or more concepts in simplified form as a prelude to the detailed description below.

Aspects of the present invention are directed to personal care compositions and, particularly personal care compositions formulated to preserve pigment associated with a tattoo contained in the skin, to improve the health of skin, and/or to improve the appearance of skin. In accordance with an aspect of the invention, provided is a personal care composition comprising from about 0.01 to about 5 wt. % of one or more vitamin E, and from about 0.01 to about 10 wt. % of one or more vitamin B comprising niacinamide, wherein all weight percentages are based on the total weight of the personal care composition.

In accordance with another aspect of the invention, provided is a personal care composition comprising from about 0.01 to about 5 wt. % of one or more vitamin E, and from about 0.01 to about 10 wt. % of one or more vitamin C comprising ascorbyl phosphate, wherein all weight percentages are based on the total weight of the personal care composition.

A list of non-limiting, exemplary embodiments is provided below:

In accordance with embodiment 1, provided is a personal care composition comprising:

a cosmetically acceptable carrier; and

a color protection component comprising:

from about 0.01 to about 5 wt. % of one or more vitamin E,

from about 0.01 to about 7 wt. % of one or more vitamin B comprising niacinamide;

wherein all weight percentages are based on the total weight of the personal care composition.

In accordance with embodiment 2, provided is a personal care composition comprising:

a cosmetically acceptable carrier; and

a color protection component comprising:

from about 0.01 to about 5 wt. % of one or more vitamin E,

from about 0.01 to about 5 wt. % of one or more vitamin C comprising ascorbyl phosphate;

wherein all weight percentages are based on the total weight of the personal care composition.

In accordance with embodiment 3, provided is a personal care composition according to embodiment 1 or embodiment 2, wherein the one or more vitamin E comprises vitamin E acetate, vitamin E succinate, alpha-tocopherol, beta-tocopherol, gamma-tocopherol, delta-tocopherol, alpha-tocotrienol, beta-tocotrienol, gamma-tocotrienol, delta-tocotrienol, tocopherol acetate, tocopherol phosphate, or any combination thereof.

In accordance with embodiment 4, provided is a personal care composition according to any foregoing embodiment, wherein the one or more vitamin E comprises alpha-tocopherol and vitamin E acetate.

In accordance with embodiment 5, provided is a personal care composition according to embodiment 1, wherein the personal care composition has a weight ratio of the total amount of vitamin E to niacinamide of from about 2:1 to about 1:6.

In accordance with embodiment 6, provided is a personal care composition according to embodiment 5, wherein the weight ratio of the total amount of vitamin E to niacinamide is from about 1:1 to about 1:3.

In accordance with embodiment 7, provided is a personal care composition according to embodiment 2, wherein the personal care composition has a weight ratio of the total amount of vitamin E to ascorbyl phosphate of from about 14:1 to about 1:2.

In accordance with an embodiment 8, provided is a personal care composition according to embodiment 7, wherein the has a weight ratio of the total amount of vitamin E to ascorbyl phosphate is from about 7:1 to about 3:1.

In accordance with embodiment 9, provided is a personal care composition according to any foregoing embodiment further comprising from about 0.5 to about 9 wt. % of lactic acid and/or a salt thereof.

In accordance with embodiment 10, provided is a personal care composition according to any foregoing embodiment further comprising from about 0.5 to about 9 wt. % of an oil.

In accordance with embodiment 11, provided is a personal care composition according to embodiment 10, wherein the oil comprises flax seed oil, castor oil, or a combination thereof.

In accordance with embodiment 12, provided is a personal care composition according to any foregoing embodiment further comprising from about 5 to about 45 wt. % of one or more surfactants.

In accordance with embodiment 13, provided is a personal care composition according to embodiment 12, wherein the one or more surfactant comprises a betaine surfactant.

In accordance with embodiment 14, provided is a personal care composition according to embodiment 13, wherein the betaine surfactant comprises cocamidopropyl betaine.

In accordance with embodiment 15, provided is a personal care composition according to embodiment 1, wherein the color protection component further comprises from about 0.01 to about 10 wt. % of one or more vitamin C.

In accordance with embodiment 16, provided is a personal care composition according to embodiment 15, wherein the one or more vitamin C is selected from ascorbyl phosphate, ascorbyl glucoside, and a combination of two or more thereof.

In accordance with embodiment 17, provided is a personal care composition according to embodiment 2, wherein the color protection component further comprises from about 0.01 to about 10 wt. % of one or more vitamin B.

In accordance with embodiment 18, provided is a personal care composition according to embodiment 17, wherein the one or more vitamin B is a vitamin B3.

In accordance with embodiment 19, provided is a personal care composition according to embodiment 17 or embodiment 18, wherein the one or more vitamin B comprises niacinamide.

In accordance with embodiment 20, provided is a personal care composition according to any foregoing embodiment, wherein the personal care composition reduces the amount of color change by at least 25 wt. %.

In accordance with embodiment 21, provided is a personal care composition according to embodiment 20, wherein the color change is assessed by a malondialdehyde evaluation relative to a placebo, wherein the placebo is free of vitamin B, vitamin C, and vitamin E.

In accordance with embodiment 22, provided is a personal care composition according any foregoing embodiment, wherein the personal care composition reduces the amount of color change by at least 25 wt. % as assessed by the difference between the amount of a light having a wavelength of 428 nm adsorbed by CI 19140 sample containing personal care composition before the irradiation with UVA+UVB and after irradiation with UVA+UVB, as compared to the difference between the amount of a light having a wavelength of 428 nm adsorbed by a CI 19140 sample containing placebo personal care composition before irradiation with UVA+UVB and after irradiation with UVA+UVB, wherein the placebo has a similar formulation as the personal care composition, but does not include the active ingredients (e.g., free of vitamin B, vitamin C, and vitamin E).

In accordance with embodiment 23, provided is a personal care composition according to any foregoing embodiment, wherein the personal care composition is in the form of a rinse off composition.

In accordance with embodiment 24, provided is a personal care composition according to any foregoing embodiment, wherein the personal care composition is in the form of a leave on composition.

### BRIEF DESCRIPTION OF THE DRAWING

FIG. 1 is a bar graph depicting the reduction in degradation of pigments associated with tattoos after application of a non-limiting exemplary composition and exposure to UV in accordance with an aspect of the invention.

### DETAILED DESCRIPTION

For illustrative purposes, the principles of the present invention are described by referencing various exemplary embodiments thereof. Although certain embodiments of the invention are specifically described herein, one of ordinary skill in the art will readily recognize that the same principles are equally applicable to and can be employed in other compositions and methods. Before explaining the disclosed embodiments of the present invention in detail, it is to be understood that the invention is not limited in its application to the details of any particular embodiment disclosed herein. The terminology used herein is for the purpose of description and not of limitation.

As used herein and in the appended claims, the singular forms “a”, “an”, and “the” include plural references unless the context dictates otherwise. The singular form of any class of the ingredients refers not only to one chemical species within that class, but also to a mixture of those chemical species. The terms “a” (or “an”), “one or more” and “at least one” may be used interchangeably herein. The terms “comprising”, “including”, and “having” may be used interchangeably. The term “include” should be interpreted as “include, but are not limited to”. The term “including” should be interpreted as “including, but are not limited to”.

As used throughout, ranges are used as shorthand for describing each and every value that is within the range. Any value within the range can be selected as the terminus of the range. Thus, a range from 1-5, includes specifically 1, 2, 3, 4 and 5, as well as subranges such as 2-5, 3-5, 2-3, 2-4, 1-4, etc.

The term “about” when referring to a number means any number within a range of 10% of the number. For example, the phrase “about 2 wt. %” refers to a number between and including 1.8 wt. % and 2.2 wt. %.

All references cited herein are hereby incorporated by reference in their entireties. In the event of a conflict in a definition in the present disclosure and that of a cited reference, the present disclosure controls.

The abbreviations and symbols as used herein, unless indicated otherwise, take their ordinary meaning. The abbreviation “wt. %” means percent by weight with respect to the personal care composition. The symbol “°” refers to a degree, such as a temperature degree or a degree of an angle. The symbols “h”, “min”, “mL”, “nm”, and “um” refer to hour, minute, milliliter, nanometer, and micrometer, respectively. The abbreviation “UV-VIS” referring to a spectrometer or spectroscopy, means Ultraviolet-Visible. The abbreviation “rpm” means revolutions per minute.

When referring to chemical structures, and names, the symbols “C”, “H”, and “O” mean carbon, hydrogen, and oxygen, respectively. The symbols “—”, “═” and “≡” mean single bond, double bond, and triple bond, respectively.

“Volatile”, as used herein, means having a flash point of less than about 100° C. “Non-volatile”, as used herein, means having a flash point of greater than about 100° C.

Any member in a list of species that are used to exemplify or define a genus, may be mutually different from, or overlapping with, or a subset of, or equivalent to, or nearly the same as, or identical to, any other member of the list of species. Further, unless explicitly stated, such as when reciting a Markush group, the list of species that define or exemplify the genus is open, and it is given that other species may exist that define or exemplify the genus just as well as, or better than, any other species listed.

The phrases, “a mixture thereof,” “a combination thereof,” or a combination of two or more thereof” do not require that the mixture include all of A, B, C, D, E, and F (although all of A, B, C, D, E, and F may be included). Rather, it indicates that a mixture of any two or more of A, B, C, D, E, and F can be included. In other words, it is equivalent to the phrase “one or more elements selected from the group consisting of A, B, C, D, E, F, and a mixture of any two or more of A, B, C, D, E, and F.” Likewise, the term “a salt thereof” also relates to “salts thereof.” Thus, where the disclosure refers to “an element selected from the group consisting of A, B, C, D, E, F, a salt thereof, and a mixture thereof,” it indicates that one or more of A, B, C, D, and F may be included, one or more of a salt of A, a salt of B, a salt of C, a salt of D, a salt of E, and a salt of F may be included, or a mixture of any two of A, B, C, D, E, F, a salt of A, a salt of B, a salt of C, a salt of D, a salt of E, and a salt of F may be included.

All components and elements positively set forth in this disclosure can be negatively excluded from the claims. In other words, the personal care compositions of the present disclosure can be free or essentially free of all components and elements positively recited throughout the present disclosure. In some instances, the personal care compositions of the present disclosure may be substantially free of non-incidental amounts of the ingredient(s) or compound(s) described herein. A non-incidental amount of an ingredient or compound is the amount of that ingredient or compound that is added into the personal care composition by itself. For example, a personal care composition may be substantially free of a non-incidental amount of an ingredient or compound, although such ingredient(s) or compound(s) may be present as part of a raw material that is included as a blend of two or more compounds.

Some of the various categories of components identified may overlap. In such cases where overlap may exist and the personal care composition includes both components (or the composition includes more than two components that overlap), an overlapping compound does not represent more than one component. For example, certain compounds may be characterized as both an emulsifier and a surfactant. If a particular personal care composition includes both an emulsifier and a surfactant, the compounds that may be characterized as both an emulsifier and a surfactant will serve only as either the emulsifier or the surfactant—not both.

For readability purposes, the chemical functional groups are in their adjective form; for each of the adjective, the word “group” is assumed. For example, the adjective “alkyl” without a noun thereafter, should be read as “an alkyl group”.

Aspects of the present invention are directed to personal care compositions formulated to improve the health of skin, the appearance of skin, and/or preserve pigment associated with a skin tattoo. In accordance with an aspect of the invention, provided is a personal care composition comprising from about 0.01 to about 5 wt. % of one or more vitamin E, and from about 0.01 to about 10 wt. % of one or more vitamin B comprising niacinamide, wherein all weight percentages are based on the total weight of the personal care composition.

In accordance with another aspect of the invention, provided is a personal care composition comprising from about 0.01 to about 5 wt. % of one or more vitamin E, and from about 0.01 to about 10 wt. % of one or more vitamin C comprising ascorbyl phosphate, wherein all weight percentages are based on the total weight of the personal care composition.

The personal care compositions may be adapted to protect and/or preserve pigments associated with tattoos (also referred to as tattoo pigments), such as Tartrazine CI 19140. Preferably, the personal care compositions reduce the amount of degradation of tattoo pigments caused by exposure to ultra violet (UV) light, such as an irradiation of UVA and UVB light, and/or chemical compounds, such as peroxides. For instance, the personal care compositions may reduce the amount of degradation of tattoo pigments caused by exposure to ultra violet (UV) light (e.g., an irradiation of 60 J/cm² of UVA and UVB light) by at least 25%, preferably about 30% or more, preferably about 34% or more, preferably about 38% or more, preferably about 40% or more, preferably about 42% or more, preferably about 44% or more, preferably about 46% or more, or preferably about 48% or more. In some embodiments, the personal care compositions may reduce the amount of degradation of tattoo pigments caused by exposure to light at an irradiation of 60 J/cm² (both UVA and UVB light) for about 1 hour, about 3 hours, about 6 hours, or about 8 hours by at least 25%, preferably about 30% or more, preferably about 34% or more, preferably about 38% or more, preferably about 40% or more, preferably about 42% or more, preferably about 44% or more, preferably about 46% or more, or preferably about 48% or more.

The reduction in the amount of oxidative species provided by the personal care compositions disclosed herein can be measured by a malondialdehyde evaluation test relative to a placebo having the same formulation, but does not include active ingredients for preserving tattoo pigments (e.g., free of vitamin B, vitamin C, and vitamin E). Quantification of malondialdehyde (MDA) formed following UV irradiation or exposure to other forms of oxidative stress, may be used as a measure of lipid peroxidation. This method has been used in a variety of in vitro, ex vivo and in vivo settings, as a measure of oxidative stress. (See, e.g., Alonso, C. (2009) An ex vivo methodology to assess the lipid peroxidation in stratum corneum. J. Photochem. Photobiol. B: Biology 97:71-76; Moysan, A. (1993) Ultraviolet A-Induced Lipid Peroxidation and Antioxidant Defense Systems in Cultured Human Skin Fibroblasts. J. Invest. Dermatol. 100:692-698; and Peres, P.S. (2011) Photoaging and chronological aging profile: Understanding oxidation of the skin. J. Photochem. Photobiol. B: Biology 103:93-97. Materials or technologies that decrease the amount of lipid peroxidation in this assay, compared to placebo, would be considered to have antioxidant properties, and predicted to help prevent fading of UV degradable pigments.

Additionally or alternatively, the reduction in the amount of degradation of tattoo pigments provided by the personal care compositions disclosed herein can be measured by assessing the difference between the amount of a light having a wavelength of 428 nm adsorbed by CI 19140 sample containing personal care composition before the irradiation with UVA+UVB and after irradiation with UVA+UVB, as compared to the difference between the amount of a light having a wavelength of 428 nm adsorbed by a CI 19140 sample containing placebo personal care composition before the irradiation with UVA+UVB and after irradiation with UVA+UVB, wherein the placebo has a similar formulation as the personal care composition, but does not include the active ingredients (e.g., free of vitamin B, vitamin C, and vitamin E).

Suitable components, such as those listed below, may be included or excluded from the formulations for the personal care compositions depending on the specific combination of other components, the form of the personal care compositions, and/or the use of the formulation (e.g., a lotion, a cream, a gel, a serum, a spray, etc.). The personal care composition may be a personal care composition, such as a skin care composition in the form of a rinse off body wash, a rinse off soap, a leave on lotion, or a leave on cream.

Rinse off compositions are typically adapted to be rinsed off using water (e.g., by washing one's hands, during a shower, or the like) after application of such personal care composition. The rinse off composition may be formulated to be rinsed off within about 1 second to about 3 minutes, about 1 second to about 2 minutes, about 1 second to about 1.5 minutes, about 1 second to about 1 minute, about 1 second to about 45 seconds, about 1 second to about 30 seconds, about 1 second to about 15 seconds; from about 30 seconds to about 3 minutes, about 30 seconds to about 2 minutes, about 30 seconds to about 1.5 minutes, about 30 seconds to about 1 minute, about 30 seconds to about 45 seconds; from about 1 minute to about 3 minutes, about 1 minute to about 2 minutes, about 1 minute to about 1.5 minutes, or any range or subrange thereof. In some instances, the rinse off composition may be rinsed off substantially immediately or immediately after application, e.g., during a shower when applied under running water.

Leave on compositions are typically adapted to remain on a keratin substrate, such as skin, for a period of time, e.g., about 3 or more minutes, about 5 or more minutes, about 10 or more minutes, about 20 or more minutes, about 30 or more minutes.

The personal care compositions typically comprise one or more vitamin E, preferably in an effective amount. The total amount of vitamin E present in the personal care compositing may be from about 0.01 to about 5 wt. %, based on the total weight of the personal care composition. For example, the personal care composition may include one or more vitamin E in an amount from about 0.01 to about 2 wt. %, about 0.01 to about 1.7 wt. %, about 0.01 to about 1.4 wt. %, about 0.01 to about 1.2 wt. %, about 0.01 to about 1 wt. %, about 0.01 to about 0.8 wt. %, about 0.01 to about 0.6 wt. %, about 0.01 to about 0.4 wt. %, about 0.01 to about 0.2 wt. %, about 0.01 to about 0.1 wt. %, about 0.01 to about 0.05 wt. %; from about 0.05 to about 3 wt. %, about 0.05 to about 2 wt. %, about 0.05 to about 1.7 wt. %, about 0.05 to about 1.4 wt. %, about 0.05 to about 1.2 wt. %, about 0.05 to about 1 wt. %, about 0.05 to about 0.8 wt. %, about 0.05 to about 0.6 wt. %, about 0.05 to about 0.4 wt. %, about 0.05 to about 0.2 wt. %, about 0.05 to about 0.1 wt. %; from about 0.1 to about 3 wt. %, about 0.1 to about 2 wt. %, about 0.1 to about 1.7 wt. %, about 0.1 to about 1.4 wt. %, about 0.1 to about 1.2 wt. %, about 0.1 to about 1 wt. %, about 0.1 to about 0.8 wt. %, about 0.1 to about 0.6 wt. %, about 0.1 to about 0.4 wt. %, about 0.1 to about 0.2 wt. %; from about 0.3 to about 3 wt. %, about 0.3 to about 2 wt. %, about 0.3 to about 1.7 wt. %, about 0.3 to about 1.4 wt. %, about 0.3 to about 1.2 wt. %, about 0.3 to about 1 wt. %, about 0.3 to about 0.8 wt. %, about 0.3 to about 0.6 wt. %; from about 0.6 to about 3 wt. %, about 0.6 to about 2 wt. %, about 0.6 to about 1.7 wt. %, about 0.6 to about 1.4 wt. %, about 0.6 to about 1.2 wt. %, about 0.6 to about 1 wt. %; from about 1 to about 3 wt. %, about 1 to about 2 wt. %, about 1 to about 1.7 wt. %, about 1 to about 1.4 wt. %, about 1 to about 1.2 wt. %; from about 1.5 to about 3 wt. %, about 1.5 to about 2 wt. %, about 2 to about 3 wt. %, or any range or subrange thereof, based on the total weight of the personal care composition.

As used herein, unless otherwise specified, “vitamin E” refers to a family of four isomers of tocopherols and four isomers of tocotrienols. All eight isomers of vitamin E have a 6-chromanol ring structure and a side chain. The four tocopherols include fully saturated side chains and include alpha-tocopherol, gamma-tocopherol, beta-tocopherol, and delta-tocopherol. The four tocotrienols include unsaturated side chains and include alpha-tocotrienol, gamma-tocotrienol, beta-tocotrienol, and delta-tocotrienol. As used herein, unless otherwise specified, the term “vitamin E” may refer to any one or more of the eight isomers. For example, as used herein, vitamin E may be or include one or more of alpha-tocopherol, gamma-tocopherol, beta-tocopherol, delta-tocopherol, alpha-tocotrienol, gamma-tocotrienol, beta-tocotrienol, delta-tocotrienol, or any combination thereof. In at least one implementation, the vitamin E includes at least one of the four tocopherols. It should be appreciated that the vitamin E and/or the isomers thereof may be or include natural forms of vitamin E, synthetic forms of vitamin E, or combinations thereof. Any one or more of the isomers of vitamin E may be in the “d” form, the “1” form, or combinations thereof. In some embodiments, vitamin E is vitamin E acetate or vitamin E succinate. In some embodiments, vitamin E is vitamin E acetate.

The vitamin E may be selected from alpha-tocopherol, beta-tocopherol, gamma-tocopherol, delta-tocopherol, alpha-tocotrienol, beta-tocotrienol, gamma-tocotrienol, delta-tocotrienol, tocopherol acetate, tocopherol phosphate, natural extracts enriched in vitamin E derivatives, and combinations of two or more thereof. In some cases, the one or more vitamin E is selected from vitamin E acetate, vitamin E succinate, alpha-tocopherol, beta-tocopherol, gamma-tocopherol, delta-tocopherol, alpha-tocotrienol, beta-tocotrienol, gamma-tocotrienol, delta-tocotrienol, tocopherol acetate, tocopherol phosphate, or any combination thereof. Preferably, at least one vitamin E is selected from alpha-tocopherol, vitamin E acetate, and a combination thereof.

The personal care composition may include two or more vitamin E compounds, such as two, three, four, five, six, seven, or any range formed thereof, of vitamin E compounds. Preferably, at least one of the two or more vitamin E is selected from alpha-tocopherol, vitamin E acetate, vitamin E succinate, delta-tocopherol, and a combination thereof. In at least one embodiment, the personal care composition includes alpha-tocopherol and vitamin E acetate.

In addition to one or more vitamin E, the personal care composition typically comprises at least one vitamin B and/or at least one vitamin C. In some preferred embodiments, the personal care composition comprises one or more vitamin B, preferably in an effective amount. In some instances, the one or more vitamin B may be present in the personal care composition in an amount from about 0.01 to about 7 wt. %, based on the total weight of the personal care composition. For example, the personal care composition may include one or more vitamin B in an amount from about 0.01 to about 3 wt. %, about 0.01 to about 2 wt. %, about 0.01 to about 1.7 wt. %, about 0.01 to about 1.4 wt. %, about 0.01 to about 1.2 wt. %, about 0.01 to about 1 wt. %, about 0.01 to about 0.8 wt. %, about 0.01 to about 0.6 wt. %, about 0.01 to about 0.4 wt. %, about 0.01 to about 0.2 wt. %, about 0.01 to about 0.1 wt. %, about 0.01 to about 0.05 wt. %; from about 0.05 to about 3 wt. %, about 0.05 to about 2 wt. %, about 0.05 to about 1.7 wt. %, about 0.05 to about 1.4 wt. %, about 0.05 to about 1.2 wt. %, about 0.05 to about 1 wt. %, about 0.05 to about 0.8 wt. %, about 0.05 to about 0.6 wt. %, about 0.05 to about 0.4 wt. %, about 0.05 to about 0.2 wt. %, about 0.05 to about 0.1 wt. %; from about 0.1 to about 3 wt. %, about 0.1 to about 2 wt. %, about 0.1 to about 1.7 wt. %, about 0.1 to about 1.4 wt. %, about 0.1 to about 1.2 wt. %, about 0.1 to about 1 wt. %, about 0.1 to about 0.8 wt. %, about 0.1 to about 0.6 wt. %, about 0.1 to about 0.4 wt. %, about 0.1 to about 0.2 wt. %; from about 0.3 to about 3 wt. %, about 0.3 to about 2 wt. %, about 0.3 to about 1.7 wt. %, about 0.3 to about 1.4 wt. %, about 0.3 to about 1.2 wt. %, about 0.3 to about 1 wt. %, about 0.3 to about 0.8 wt. %, about 0.3 to about 0.6 wt. %; from about 0.6 to about 3 wt. %, about 0.6 to about 2 wt. %, about 0.6 to about 1.7 wt. %, about 0.6 to about 1.4 wt. %, about 0.6 to about 1.2 wt. %, about 0.6 to about 1 wt. %; from about 1 to about 7 wt. %, about 1 to about 5 wt. %, about 1 to about 3 wt. %, about 1 to about 2 wt. %, about 1 to about 1.7 wt. %, about 1 to about 1.4 wt. %, about 1 to about 1.2 wt. %; from about 1.5 to about 7 wt. %, about 1.5 to about 5 wt. %, about 1.5 to about 4 wt. %, about 1.5 to about 3 wt. %, about 1.5 to about 2 wt. %, from about 2 to about 7 wt. %, about 2 to about 5 wt. %, about 2 to about 4 wt. %, about 2 to about 3 wt. %, from about 3 to about 7 wt. %, about 3 to about 5 wt. %, about 3 to about 4 wt. %, or any range or subrange thereof, based on the total weight of the personal care composition.

The one or more vitamin B of the personal care composition may be selected from vitamin B₃. The vitamin B₃ may have a structure in accordance with the below formula:

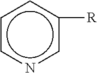

wherein R is —CONH₂ (i.e., niacinamide), —COOH (i.e., nicotinic acid) or —CH₂OH (i.e., nicotinyl alcohol); a derivative thereof, and/or a salt thereof. The vitamin B may be selected from deritives of vitamin B.

Examples of vitamin B₃ include those selected from nicotinic acid esters, non-vasodilating esters of nicotinic acid, nicotinyl amino acids, nicotinyl alcohol esters of carboxylic acids (e.g., straight-chain or branched chain, cyclic or acyclic, saturated or unsaturated (including aromatic), and substituted or unsubstituted, nicotinic acid N-oxide and niacinamide N-oxide, tocopherol nicotinate and inositol hexanicotinate. Examples of derivatives of niacinamide include nicotinyl amino acids, derived, e.g., from the reaction of an activated nicotinic acid compound (e.g., nicotinic acid azide or nicotinyl chloride) with an amino acid, and nicotinyl alcohol esters of organic carboxylic acids (e.g., C₁-C₁₈). Specific examples of such derivatives include nicotinuric acid and nicotinyl hydroxamic acid, which have the following chemical structures:

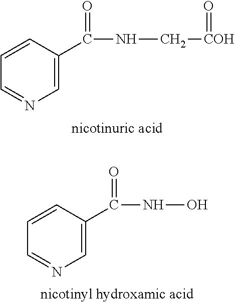

Additional derivatives of niacinamide includes nicotinyl alcohol esters, such as nicotinyl alcohol esters of the carboxylic acids, salicylic acid, acetic acid, glycolic acid, palmitic acid and the like. Further examples of derivatives of niacinamide include 2-chloronicotinamide, 6-aminonicotinamide, 6-methylnicotinamide, n-methyl-nicotinamide, n,n-diethylnicotinamide, n-(hydroxymethyl)-nicotinamide, quinolinic acid imide, nicotinanilide, n-benzylnicotinamide, n-ethylnicotinamide, nifenazone, nicotinaldehyde, isonicotinic acid, methyl isonicotinic acid, thionicotinamide, nialamide, 1-(3-pyridylmethyl) urea, 2-mercaptonicotinic acid, nicomol, niaprazine, and combinations thereof.

In at least one embodiment, the personal care composition includes one or more vitamin B comprising, consisting essentially of, or consisting of niacinamide.

The personal care composition may include one or more vitamin C, preferably in an effective amount. The total amount of vitamin C present in the personal care composition may be from about 0.01 to about 5 wt. %, based on the total weight of the personal care composition. For example, the personal care composition may include one or more vitamin C in an amount from about 0.01 to about 2 wt. %, about 0.01 to about 1.7 wt. %, about 0.01 to about 1.4 wt. %, about 0.01 to about 1.2 wt. %, about 0.01 to about 1 wt. %, about 0.01 to about 0.8 wt. %, about 0.01 to about 0.6 wt. %, about 0.01 to about 0.4 wt. %, about 0.01 to about 0.2 wt. %, about 0.01 to about 0.1 wt. %, about 0.01 to about 0.05 wt. %; from about 0.05 to about 3 wt. %, about 0.05 to about 2 wt. %, about 0.05 to about 1.7 wt. %, about 0.05 to about 1.4 wt. %, about 0.05 to about 1.2 wt. %, about 0.05 to about 1 wt. %, about 0.05 to about 0.8 wt. %, about 0.05 to about 0.6 wt. %, about 0.05 to about 0.4 wt. %, about 0.05 to about 0.2 wt. %, about 0.05 to about 0.1 wt. %; from about 0.1 to about 3 wt. %, about 0.1 to about 2 wt. %, about 0. 1 to about 1.7 wt. %, about 0.1 to about 1.4 wt. %, about 0.1 to about 1.2 wt. %, about 0.1 to about 1 wt. %, about 0.1 to about 0.8 wt. %, about 0.1 to about 0.6 wt. %, about 0.1 to about 0.4 wt. %, about 0.1 to about 0.2 wt. %; from about 0.3 to about 3 wt. %, about 0.3 to about 2 wt. %, about 0.3 to about 1.7 wt. %, about 0.3 to about 1.4 wt. %, about 0.3 to about 1.2 wt. %, about 0.3 to about 1 wt. %, about 0.3 to about 0.8 wt. %, about 0.3 to about 0.6 wt. %; from about 0.6 to about 3 wt. %, about 0.6 to about 2 wt. %, about 0.6 to about 1.7 wt. %, about 0.6 to about 1.4 wt. %, about 0.6 to about 1.2 wt. %, about 0.6 to about 1 wt. %; from about 1 to about 3 wt. %, about 1 to about 2 wt. %, about 1 to about 1.7 wt. %, about 1 to about 1.4 wt. %, about 1 to about 1.2 wt. %; from about 1.5 to about 3 wt. %, about 1.5 to about 2 wt. %, about 2 to about 3 wt. %, or any range or subrange thereof, based on the total weight of the personal care composition.

As used herein, “vitamin C” may be ascorbic acid, derivatives thereof, and/or salts thereof. Ascorbic acid exists as two enantiomers commonly denoted “1” (for “levo”) and “d” (for “dextro”). The “1” isomer is the one most often encountered. Ascorbic acid is also referred to as L (+)-ascorbic acid or 1-ascorbic acid. The ascorbic acid derivatives may be, but are not limited to, L-ascorbic acid, calcium ascorbate, calcium 1-ascorbate dihydrate, magnesium ascorbate, potassium ascorbate, magnesium L-ascorbyl phosphate (also referred to as: magnesium ascorbate phosphate or ascorbic acid phosphate magnesium salt), L-ascorbic acid 2-phosphate sesquimagnesium salt hydrate, (+) sodium L-ascorbate, dehydro-1-(+)-ascorbic acid dimer, sodium ascorbyl phosphate (also referred to as: ascorbic acid phosphate sodium salt, sodium 1-ascorbyl phosphate, 2-phospho-L-ascorbic acid trisodium salt, L-ascorbic acid 2-phosphate trisodium salt or sodium L-ascorbyl-2-phosphate), ascorbic acid-2-glucoside, ascorbyl dipalmitate, ascorbyl methylsilanol pectinate, ascorbyl stearate, disodium ascorbyl sulfate, ascorbyl 6-palmitate, calcium ascorbyl phosphate, ascorbyl acetate, ascorbyl propionate, ascorbyl stearate, ascorbyl palmitate, ascorbyl dipalmitate, ascorbyl glucoside, ascorbic acid polypeptide, ethyl ascorbyl ether, ascorbyl ethyl silanol pectinate, or the like, or combinations thereof. For example, the personal care composition includes a vitamin C selected from ascorbyl glucoside, ascorbic acid-2-glucoside, ascorbyl acetate, and a combination of two or more thereof. In certain embodiments, the personal care composition comprises from ascorbyl glucoside, ascorbic acid-2-glucoside, or a combination thereof.

The personal care composition may be formulated to specifically have a weight ratio of the total amount of vitamin E (e.g., any of the vitamin E compounds listed herein) to the vitamin B (e.g., any of the vitamin B compounds listed herein) of from about 2:1 to about 1:6. For instance, the personal care composition may have a weight ratio of the total amount of vitamin E to the vitamin B of about 2:1 to about 1:5, about 2:1 to about 1:4, about 2:1 to about 1:3, about 2:1 to about 1:2; from about 1:1 to about 1:6, about 1:1 to about 1:5, about 1:1 to about 1:4, about 1:1 to about 1:3, about 1:1 to about 1:2, or any range or subrange thereof. In at least one embodiment, the weight ratio of the total amount of vitamin E to the vitamin B is about 1:2.

In some embodiments, the personal care composition may be formulated to specifically have a weight ratio of the total amount of vitamin E (e.g., any of the vitamin E compounds listed herein) to the niacinamide of from about 2:1 to about 1:6. For instance, the personal care composition may have a weight ratio of the total amount of vitamin E to the niacinamide of about 2:1 to about 1:5, about 2:1 to about 1:4, about 2:1 to about 1:3, about 2:1 to about 1:2; from about 1:1 to about 1:6, about 1:1 to about 1:5, about 1:1 to about 1:4, about 1:1 to about 1:3, about 1:1 to about 1:2, or any range or subrange thereof. In at least one embodiment, the weight ratio of the total amount of vitamin E to the niacinamide is about 1:2.

Additionally or alternatively, the personal care composition may be formulated to specifically have a weight ratio of the total amount of vitamin E (e.g., any of the vitamin E compounds listed herein) to the vitamin C (e.g., any of the vitamin C compounds listed herein) of from about 14:1 to about 1:2. In certain embodiment, the personal care composition has a weight ratio of the total amount of vitamin E to the vitamin C that is from about 14:1 to about 1:2, about 12:1 to about 1:2, about 10:1 to about 1:2, about 8:1 to about 1:2, about 7:1 to about 1:2, about 6:1 to about 1:2, about 5:1 to about 1:2, about 4:1 to about 1:2, about 3:1 to about 1:2, about 2:1 to about 1:2; from about 14:1 to about 1:1, about 12:1 to about 1:1, about 10:1 to about 1:1, about 8:1 to about 1:1, about 7:1 to about 1:1, about 6:1 to about 1:1, about 5:1 to about 1:1, about 4:1 to about 1:1, about 3:1 to about 1:2, about 2:1 to about 1:1; from about 14:1 to about 2:1, about 12:1 to about 2:1, about 10:1 to about 2:1, about 8:1 to about 2:1, about 7:1 to about 2:1, about 6:1 to about 2:1, about 5:1 to about 2:1, about 4:1 to about 2:1, about 3:1 to about 2:1; from about 14:1 to about 3:1, about 12:1 to about 3:1, about 10:1 to about 3:1, about 8:1 to about 3:1, about 7:1 to about 3:1, about 6:1 to about 3:1, about 5:1 to about 3:1, about 4:1 to about 3:1; from about 14:1 to about 4:1, about 12:1 to about 4:1, about 10:1 to about 4:1, about 8:1 to about 4:1, about 7:1 to about 4:1, about 6:1 to about 4:1, about 5:1 to about 4:1, or any range or subrange thereof.

In certain embodiments, the personal care compositions are formulated to specifically have a weight ratio of the total amount of vitamin E (e.g., any of the vitamin E compounds listed herein) to the ascorbyl phosphate of from about 14:1 to about 1:2. For example, the personal care composition may have a weight ratio of the total amount of vitamin E to the ascorbyl phosphate that is from about 14:1 to about 1:2, about 12:1 to about 1:2, about 10:1 to about 1:2, about 8:1 to about 1:2, about 7:1 to about 1:2, about 6:1 to about 1:2, about 5:1 to about 1:2, about 4:1 to about 1:2, about 3:1 to about 1:2, about 2:1 to about 1:2; from about 14:1 to about 1:1, about 12:1 to about 1:1, about 10:1 to about 1:1, about 8:1 to about 1:1, about 7:1 to about 1:1, about 6:1 to about 1:1, about 5:1 to about 1:1, about 4:1 to about 1:1, about 3:1 to about 1:2, about 2:1 to about 1:1; from about 14:1 to about 2:1, about 12:1 to about 2:1, about 10:1 to about 2:1, about 8:1 to about 2:1, about 7:1 to about 2:1, about 6:1 to about 2:1, about 5:1 to about 2:1, about 4:1 to about 2:1, about 3:1 to about 2:1; from about 14:1 to about 3:1, about 12:1 to about 3:1, about 10:1 to about 3:1, about 8:1 to about 3:1, about 7:1 to about 3:1, about 6:1 to about 3:1, about 5:1 to about 3:1, about 4:1 to about 3:1; from about 14:1 to about 4:1, about 12:1 to about 4:1, about 10:1 to about 4:1, about 8:1 to about 4:1, about 7:1 to about 4:1, about 6:1 to about 4:1, about 5:1 to about 4:1, or any range or subrange thereof. In one embodiment, the weight ratio of the total amount of vitamin E to ascorbyl phosphate is about 5:1.

The personal care compositions may comprise lactic acid and/or a salt thereof, e.g., in an amount from about 0.5 to about 9 wt. %, based on the total weight of the personal care composition. For instance, the personal care composition may comprise lactic acid and/or a salt thereof in an amount from about 0.5 to about 9 wt. %, about 0.5 to about 7 wt. %, about 0.5 to about 6 wt. %, about 0.5 to about 5 wt. %, about 0.5 to about 4 wt. %, about 0.5 to about 3 wt. %, about 0.5 to about 2 wt. %, about 0.5 to about 1.5 wt. %, about 0.5 to about 1 wt. %; from about 1 to about 9 wt. %, about 1 to about 7 wt. %, about 1 to about 6 wt. %, about 1 to about 5 wt. %, about 1 to about 4 wt. %, about 1 to about 3 wt. %, about 1 to about 2 wt. %; from about 1.5 to about 9 wt. %, about 1.5 to about 7 wt. %, about 1.5 to about 6 wt. %, about 1.5 to about 5 wt. %, about 1.5 to about 4 wt. %, about 1.5 to about 3 wt. %, about 1.5 to about 2 wt. %; from about 2 to about 9 wt. %, about 2 to about 7 wt. %, about 2 to about 6 wt. %, about 2 to about 5 wt. %, about 2 to about 4 wt. %, about 2 to about 3 wt. %; from about 3 to about 9 wt. %, about 3 to about 7 wt. %, about 3 to about 6 wt. %, about 3 to about 5 wt. %, about 3 to about 4 wt. %; from about 4 to about 9 wt. %, about 4 to about 7 wt. %, about 4 to about 6 wt. %, about 4 to about 5 wt. %, or any range or subrange thereof, based on the total weight of the personal care composition. The personal care composition may contain the lactic acid in the form of salt, such as sodium lactate, potassium lactate, and/or combinations thereof.

The personal care composition may comprise a hydrophobic component, a hydrophilic component, or a combination thereof. The hydrophilic component may include a monoalcohol, a polyol, a glycol, or a combination of two or more thereof. The hydrophobic component may comprise an oil, a wax, a silicone, or a mixture thereof. The oil or wax may be a linear or branched hydrocarbon, optionally of mineral or synthetic origin. The linear or branched hydrocarbon may be selected from volatile or non-volatile liquid paraffins and derivatives thereof, petroleum jelly, polydecenes, isohexadecane, isododecane, hydrogenated polyisobutene, a mixture of n-undecane (C₁₁) and of n-tridecane (C₁₃), and a combination of two or more thereof. Examples of liquid hydrocarbons include linear and branched oils, such as liquid paraffin, squalene, squalane, mineral oil, and low viscosity synthetic hydrocarbons (e.g., polyalphaolefins and polybutene).

In some cases, the oil is a plant-based oil. For example, the oil may be a plant-based oil selected from sunflower oil, corn oil, soybean oil, marrow oil, grapeseed oil, sesame seed oil, hazelnut oil, apricot oil, macadamia oil, araba oil, coriander oil, castor oil, flax seed oil, avocado oil, jojoba oil, shea butter oil, and a combination of two or more thereof.

The one or more oil(s) may include one or more volatile or non-volatile silicones. For instance, a volatile silicone oil may be included in the personal care composition, optionally selected from cyclopolydimethylsiloxanes (cyclomethicones), such as cyclohexadimethylsiloxane and cyclopentadimethylsiloxane. Other useful skin and/or compatible oils may be silicone based, for example, linear and cyclic polydimethyl siloxane, organo functional silicones (alkyl and alkyl aryl), and amino silicones.

The silicone may have from 2 to 24 carbon atoms. For example, the silicone may be a polydimethylsiloxane, optionally having from 2 to 24 carbon atoms, from 2 to 20 carbon atoms, or from 6 to 20 carbon atoms. In some instances, the silicone comprises a phenyl silicones, optionally selected from phenyl trimethicones, phenyl dimethicones, phenyltrimethylsiloxydiphenylsiloxanes, diphenyl dimethicones, diphenylmethyldiphenyltrisiloxanes, 2-phenylethyl trimethylsiloxy silicates, polymethylphenylsiloxanes, and a combination of two or more thereof. Non-limiting examples of silicones include dimethiconol, dimethicone, and a mixture thereof. Non-limiting examples of amino silicone oils include amodimethicone, aminoethylaminopropyl dimethicone, aminopropyl dimethicone, and a mixture thereof.

The total amount of the hydrophobic component and/or the oil present in the personal care composition may be, e.g., from about 1 to about 35 wt. %, based on the total weight of the personal care composition. For instance, the personal care composition may include the hydrophobic component and/or the oil(s) in an amount from about 1 to about 20 wt. %, about 1 to about 10 wt. %, about 1 to about 8 wt. %, about 1 to about 6 wt. %, about 1 to about 4 wt. %, about 1 to about 3 wt. %; from about 3 to about 20 wt. %, about 3 to about 10 wt. %, about 3 to about 8 wt. %, about 3 to about 6 wt. %; from about 5 to about 30 wt. %, about 5 to about 25 wt. %, about 5 to about 21 wt. %, about 5 to about 17 wt. %, about 5 to about 14 wt. %, about 5 to about 11 wt. %; from about 10 to about 35 wt. %, about 10 to about 30 wt. %, about 10 to about 25 wt. %, about 10 to about 21 wt. %, about 10 to about 17 wt. %, about 10 to about 14 wt. %; from about 15 to about 35 wt. %, about 15 to about 30 wt. %, about 15 to about 25 wt. %, about 15 to about 21 wt. %, about 15 to about 19 wt. %; from about 18 to about 35 wt. %, about 18 to about 30 wt. %, about 18 to about 25 wt. %, about 18 to about 21 wt. %; from about 21 to about 35 wt. %, about 21 to about 30 wt. %, about 21 to about 25 wt. %; from about 24 to about 35 wt. %, about 24 to about 30 wt. %; from about 27 to about 35 wt. %, about 27 to about 30 wt. %, or any range or subrange thereof, based on the total weight of the personal care composition.

The personal care composition may comprise one or more surfactant(s). The one or more surfactant(s) can be any anionic, nonionic, amphoteric, zwitterionic surfactant, or combinations of two or more thereof. The amount of surfactant(s) in the composition may be about 5 to about 45 wt. %, based on the total weight of the personal care composition. For example, the amount of surfactant(s) present in the personal care composition may be from about 5 to about 45 wt. %, about 5 to about 35 wt. %, about 5 to about 25 wt. %, about 5 to about 20 wt. %, about 5 to about 15 wt. %, about 5 to about 13 wt. %; from about 10 to about 45 wt. %, about 10 to about 35 wt. %, about 10 to about 25 wt. %, about 10 to about 20 wt. %, about 10 to about 15 wt. %, about 10 to about 13 wt. %; from about 14 to about 45 wt. %, about 14 to about 35 wt. %, about 14 to about 25 wt. %, about 14 to about 20 wt. %, about 14 to about 15 wt. %, about 14 to about 13 wt. %; from about 17 to about 45 wt. %, about 17 to about 35 wt. %, about 17 to about 25 wt. %, about 17 to about 20 wt. %, about 17 to about 15 wt. %; from about 20 to about 45 wt. %, about 20 to about 35 wt. %, about 20 to about 25 wt. %, about 20 to about 20 wt. %, about 20 to about 15 wt. %, about 20 to about 13 wt. %; from about 25 to about 45 wt. %, about 25 to about 35 wt. %; from about 30 to about 45 wt. %, about 30 to about 35 wt. %, about 30 to about 25 wt. %, about 30 to about 20 wt. %; from about 35 to about 45 wt. %, about 35 to about 40 wt. %, including all ranges and subranges therebetween, based on the total weight of the personal care composition.

Under an embodiment, multiple surfactants are used to achieve desired product qualities. For example, a primary surfactant may be used to provide good foaming ability and cleaning effectiveness, while a secondary surfactant adds qualities of mildness to prevent irritation or over-drying of the skin. A variety of anionic surfactants can be utilized in the personal care composition including, e.g., long chain alkyl (C₆-C₂₂) materials such as long chain alkyl sulfates, long chain alkyl sulfonates, long chain alkyl phosphates, long chain alkyl ether sulfates, long chain alkyl alpha olefin sulfonates, long chain alkyl taurates, long chain alkyl isethionates (SCI), long chain alkyl glyceryl ether sulfonates (AGES), sulfosuccinates, and the like. These anionic surfactants can be alkoxylated, for example, ethoxylated, although alkoxylation is not required. For instance, the anionic surfactant may be a C₁₂₋₁₄ ethoxylated alcohol sulfate.

The anionic surfactants may be associated with a cation ion selected from sodium, potassium, alkyl and ammonium, an alkanol ammonium in their salt form. Other equivalent anionic surfactants may be used.

The personal care composition may include one or more anionic surfactant in any of the amounts or ranges disclosed above with respect to the amount of surfactant. In some instance, the one or more anionic surfactant may be present in an amount from about 2 to about 15 wt. %, about 2 to about 13 wt. %, about 2 to about 11 wt. %, about 2 to about 9 wt. %, about 2 to about 7 wt. %, about 2 to about 5 wt. %; from about 4 to about 15 wt. %, about 4 to about 13 wt. %, about 4 to about 11 wt. %, about 4 to about 9 wt. %, about 4 to about 7 wt. %; from about 6 to about 15 wt. %, about 6 to about 13 wt. %, about 6 to about 11 wt. %, about 6 to about 9 wt. %; from about 8 to about 15 wt. %, about 8 to about 13 wt. %, about 8 to about 11 wt. %; from about 10 to about 15 wt. %, about 10 to about 13 wt. %, or any range or subrange thereof, based on the personal care composition.

Amphoteric surfactants may also be included in the composition. Amphoteric surfactants include, but are not limited to, derivatives of aliphatic secondary and tertiary amines in which the aliphatic radical can be straight chain or branched and wherein one of the aliphatic substituents contains about 8 to about 18 carbon atoms and one contains an anionic water solubilizing group, e.g., carboxy, sulfonate, sulfate, phosphate, or phosphonate. Examples of such compounds include sodium 3-dodecyaminopropionate, sodium 3-dodecylaminopropane sulfonate, N-alkyl taurines and N-higher alkyl aspartic acids. Other equivalent amphoteric surfactants may be used.

Additional examples of amphoteric surfactants include, but are not limited to, a range of betaines including, for example, high alkyl betaines, such as coco dimethyl carboxymethyl betaine, lauryl dimethyl carboxy-methyl betaine, lauryl dimethyl alpha-carboxyethyl betaine, cetyl dimethyl carboxymethyl betaine, lauryl bis-(2-hydroxyethyl) carboxy methyl betaine, stearyl bis-(2-hydroxypropyl) carboxymethyl betaine, oleyl dimethyl gamma-carboxypropyl betaine, and lauryl bis-(2-hydroxypropyl) alpha-carboxyethyl betaine, sulfobetaines such as coco dimethyl sulfopropyl betaine, stearyl dimethyl sulfopropyl betaine, amido betaines, amidosulfobetaines, and a combination of two or more thereof. Betaines having a long chain alkyl group, particularly coco, may be useful as are those that include an amido groups, such as the cocamidopropyl and cocoamidoethyl betaines.

The personal care composition may include one or more amphoteric surfactant(s) in any of the amounts or ranges disclosed above with respect to the amount of surfactant. In some instance, the one or more amphoteric surfactant(s) may be present in an amount from about 2 to about 15 wt. %, about 2 to about 13 wt. %, about 2 to about 11 wt. %, about 2 to about 9 wt. %, about 2 to about 7 wt. %, about 2 to about 5 wt. %; from about 4 to about 15 wt. %, about 4 to about 13 wt. %, about 4 to about 11 wt. %, about 4 to about 9 wt. %, about 4 to about 7 wt. %; from about 6 to about 15 wt. %, about 6 to about 13 wt. %, about 6 to about 11 wt. %, about 6 to about 9 wt. %; from about 8 to about 15 wt. %, about 8 to about 13 wt. %, about 8 to about 11 wt. %; from about 10 to about 15 wt. %, about 10 to about 13 wt. %, or any range or subrange thereof, based on the personal care composition.

The personal care composition may include one or more surfactant selected from nonionic surfactants. Examples of nonionic surfactants include polysorbate 20, long chain alkyl glucosides having C₈-C₂₂ alkyl groups; coconut fatty acid monoethanolamides such as cocamide MEA; coconut fatty acid diethanolamides, fatty alcohol ethoxylates (alkylpolyethylene glycols); alkylphenol polyethylene glycols; alkyl mercaptan polyethylene glycols; fatty amine ethoxylates (alkylaminopolyethylene y acid ethoxylates (acylpolyethylene glycols);

polypropylene glycol ethoxylates (for example the PLURONIC™ block copolymers commercially available from BASF); fatty acid alkylolamides, (fatty acid amide polyethylene glycols); N-alkyl-, N-alkoxypolyhydroxy fatty acid amides; sucrose esters; sorbitol esters; polyglycol ethers; and combinations thereof.

The one or more nonionic surfactant(s) may be present in an amount that may range from about 0.1 to about 7 wt. %, based on the total weight of the personal care composition. In some instances, the one or more nonionic surfactant(s) is present in the personal care composition in an amount from about 0.1 to about 6 wt. %, about 0.1 to about 5 wt. %, about 0.1 to about 4 wt. %, about 0.1 to about 3 wt. %, about 0.1 to about 2 wt. %, about 0.1 to about 1 wt. %; from about 0.4 to about 7 wt. %, about 0.4 to about 6 wt. %, about 0.4 to about 5 wt. %, about 0.4 to about 4 wt. %, about 0.4 to about 3 wt. %, about 0.4 to about 2 wt. %, about 0.4 to about 1 wt. %; from about 0.8 to about 7 wt. %, about 0.8 to about 6 wt. %, about 0.8 to about 5 wt. %, about 0.8 to about 4 wt. %, about 0.8 to about 3 wt. %, about 0.8 to about 2 wt. %; from about 1.4 to about 7 wt. %, about 1.4 to about 6 wt. %, about 1.4 to about 5 wt. %, about 1.4 to about 4 wt. %, about 1.4 to about 3 wt. %; from about 2 to about 7 wt. %, about 2 to about 6 wt. %, about 2 to about 5 wt. %, about 2 to about 4 wt. %, about 2 to about 3 wt. %; from about 3 to about 7 wt. %, about 3 to about 6 wt. %, about 3 to about 5 wt. %; from about 4 to about 7 wt. %, about 4 to about 6 wt. %, about 5 to about 7 wt. %, or any range or subrange thereof, based on the total weight of the personal care composition

Cationic surfactants can also be included in the composition. Examples of cationic surfactants include any quaternium or polyquaternium compound. Cationic surfactants can be included at any desired level. The cationic surfactant(s) may be present in an amount from about 0.1 to about 5 wt. %, based on the total weight of the personal care composition. For example, the personal care composition may include one or more cationic surfactant(s) in an amount from about 0.1 to about 4 wt. %, about 0.1 to about 3 wt. %, about 0.1 to about 2 wt. %, about 0.1 to about 1 wt. %; from about 0.3 to about 5 wt. %, about 0.3 to about 4 wt. %, about 0.3 to about 3 wt. %, about 0.3 to about 2 wt. %, about 0.3 to about 1 wt. %; from about 0.6 to about 5 wt. %, about 0.6 to about 4 wt. %, about 0.6 to about 3 wt. %, about 0.6 to about 2 wt. %, about 0.6 to about 1 wt. %; from about 1 to about 5 wt. %, about 1 to about 4 wt. %, about 1 to about 3 wt. %, about 1 to about 2 wt. %; from about 1.5 to about 5 wt. %, about 1.5 to about 4 wt. %, about 1.5 to about 3 wt. %; from about 2 to about 5 wt. %, about 2 to about 4 wt. %, about 2 to about 3 wt. %; from about 3 to about 5 wt. %, about 3 to about 4 wt. %, about 4 to about 5 wt. %, including any range or subrange thereof, based on the total weight of the personal care composition.

The personal care compositions, in certain embodiments, may further comprises a cationic polymer. The cationic polymers can comprise mixtures of monomer units derived from amine- and/or quaternary ammonium-substituted monomer and/or compatible spacer monomers. Suitable cationic polymers include, for example: copolymers of 1-vinyl-2-pyrrolidine and 1-vinyl-3-methyl-imidazolium salt (e.g., chloride salt) (referred to as Polyquaternium-16) such as those commercially available from BASF under the LUVIQUAT tradename (e.g., LUVIQUAT FC 370); copolymers of 1-vinyl-2-pyrrolidine and dimethylaminoethyl methacrylate (referred to as Polyquaternium-11) such as those commercially from Gar Corporation (Wayne, N.J., USA) under the GAFQUAT tradename (e.g., GAFQUAT 755N); and cationic diallyl quaternary ammonium-containing polymer including, for example, dimethyldiallyammonium chloride homopolymer and copolymers of acrylamide and dimethyldiallyammonium chloride (referred to as Polyquaternium-6 and Polyquaternium-7), and combinations thereof.

Other cationic polymers that may be used include polysaccharide polymers, such as cationic cellulose derivatives and cationic starch derivatives. Cationic cellulose is available from Amerchol Corp. (Edison, N.J., USA) in their Polymer JR (trade mark) and LR (trade mark) series of polymers, as salts of hydroxyethyl cellulose reacted with trimethyl ammonium substituted epoxide (referred to as Polyquaternium-10). Another type of cationic cellulose includes the polymeric quaternary ammonium salts of hydroxyethyl cellulose reacted with lauryl dimethyl ammonium-substituted epoxide (referred to as Polyquaternium-24). These materials are available from Amerchol Corp. (Edison, N.J., USA) under the tradename Polymer LM-200. Additionally or alternatively, the cationic polymers may include cationic guar gum derivatives, such as guar hydroxypropyltrimonium chloride.

The personal care composition may include or be selected from polyquaterniums. In one instance, the one or more cationic polymers is selected from polyquaternium 4, polyquaternium 6, polyquaternium 7, polyquaternium 10, polyquaternium 11, polyquaternium 16, polyquaternium 22, polyquaternium 28, polyquaternium 32, polyquaternium-46, polyquaternium-51, polyquaternium-52, polyquaternium-53, polyquaternium-54, polyquaternium-55, polyquaternium-56, polyquaternium-57, polyquaternium-58, polyquaternium-59, polyquaternium-60, polyquaternium-63, polyquaternium-64, polyquaternium-65, polyquaternium-66, polyquaternium-67, polyquaternium-70, polyquaternium-73, polyquaternium-74, polyquaternium-75, polyquaternium-76, polyquaternium-77, polyquaternium-78, polyquaternium-79, polyquaternium-80, polyquaternium-81, polyquaternium-82, polyquaternium-84, polyquaternium-85, polyquaternium-86, polyquaternium-87, polyquaternium-90, polyquaternium-91, polyquaternium-92, polyquaternium-94, guar hydroxypropyltrimonium chloride, and a combination of two or more thereof. In at least one embodiment, the cationic polymer comprises a copolymer of acrylamide and diallyldimethylammonium chloride.

The cationic polymer(s) may be present in an amount that may range from about 0.1 to about 7 wt. %, based on the total weight of the personal care composition. For instance, the cationic polymer(s) may be present in an amount from about 0.1 to about 6 wt. %, about 0.1 to about 5 wt. %, about 0.1 to about 4 wt. %, about 0.1 to about 3 wt. %, about 0.1 to about 2 wt. %, about 0.1 to about 1 wt. %; from about 0.4 to about 7 wt. %, about 0.4 to about 6 wt. %, about 0.4 to about 5 wt. %, about 0.4 to about 4 wt. %, about 0.4 to about 3 wt. %, about 0.4 to about 2 wt. %, about 0.4 to about 1 wt. %; from about 0.8 to about 7 wt. %, about 0.8 to about 6 wt. %, about 0.8 to about 5 wt. %, about 0.8 to about 4 wt. %, about 0.8 to about 3 wt. %, about 0.8 to about 2 wt. %; from about 1.4 to about 7 wt. %, about 1.4 to about 6 wt. %, about 1.4 to about 5 wt. %, about 1.4 to about 4 wt. %, about 1.4 to about 3 wt. %; from about 2 to about 7 wt. %, about 2 to about 6 wt. %, about 2 to about 5 wt. %, about 2 to about 4 wt. %, about 2 to about 3 wt. %; from about 3 to about 7 wt. %, about 3 to about 6 wt. %, about 3 to about 5 wt. %; from about 4 to about 7 wt. %, about 4 to about 6 wt. %, about 5 to about 7 wt. %, or any range or subrange thereof, based on the total weight of the personal care composition

The personal care composition may include one or more pH adjusters to increase or decrease the overall pH of the personal care composition. For example, one or more acids may be included to decrease the pH of the personal care composition. Examples of suitable acids for decreasing the pH of the personal care composition include, but are not limited to, citric acid, acetic acid, and the like. The personal care composition may include one or more bases, such as sodium hydroxide, potassium hydroxide and the like, to increase the pH of the personal care composition. Additional or alternative acids and bases that are suitable for adjusting the pH of the personal care composition are readily known to one of ordinary skill in the art.

The amount of the pH adjuster in the personal care composition may be based on the desired pH of the final personal care composition and/or product. For example, the total amount of the pH adjuster may range from about 0.05 to about 20 wt. %, based on the total weight of the personal care composition. In some instances, the total amount of pH adjuster is from about 0.05 to about 15 wt. %, about 0.1 to about 10 wt. %, or about 0.12 to about 5 wt. %, including ranges and sub-ranges therebetween, based on the total weight of the personal care composition.

The personal care compositions may have a pH from 4.5 to about 10, 4.5 to about 9, 4.5 to about 8, 4.5 to about 7, 4.5 to about 6; from about 5 to about 10, about 5 to about 9, about 5 to about 8, about 5 to about 7, about 5 to about 6; from about 6 to about 10, about 6 to about 9, about 6 to about 8, or about 6 to about 7; from about 7 to about 10, about 7 to about 9, or about 7 to about 8, including any ranges and subranges therebetween. In some embodiments, the personal care composition has a pH of from about 3.5 to about 7.5. In other embodiments, the personal care composition has a pH of from about 4 to about 7. In further embodiments, the personal care composition has a pH of from about 4.5 to about 6.5. Still other embodiments provide personal care compositions having a pH of from about 5 to about 6.5.

Additional ingredients may be present in the personal care composition. These include water and ingredients to thicken, preserve, emulsify, add fragrance, and color. The personal care compositions may include any of the following additional ingredients in an amount of from about 0.01 to about 5 wt. %, based on the total weight of the personal care composition. In some instances, the amount of additional ingredients present in the personal care composition is from about 0.01 to about 4 wt. %, about 0.01 to about 3 wt. %, about 0.01 to about 2 wt. %, about 0.01 to about 1 wt. %, about 0.01 to about 0.5 wt. %, about 0.01 to about 0.1 wt. %; about 0.1 to about 5 wt. %, about 0.1 to about 4 wt. %, about 0.1 to about 3 wt. %, about 0.1 to about 2 wt. %, about 0.1 to about 1 wt. %, about 0.1 to about 0.5 wt. %, about 0.1 to about 0.1 wt. %; about 0.5 to about 5 wt. %, about 0.5 to about 4 wt. %, about 0.5 to about 3 wt. %, about 0.5 to about 2 wt. %, about 0.5 to about 1 wt. %; about 0.75 to about 5 wt. %, about 0.75 to about 4 wt. %, about 0.75 to about 3 wt. %, about 0.75 to about 2 wt. %, about 0.75 to about 1 wt. %; about 1 to about 5 wt. %, about 1 to about 4 wt. %, about 1 to about 3 wt. %, about 1 to about 2 wt. %; about 2 to about 5 wt. %, about 2 to about 4 wt. %, about 2 to about 3 wt. %; about 3 to about 5 wt. %, about 3 to about 4 wt. %, including any range or subrange therebetween, based on the total weight of the personal care composition.

The personal care composition can comprise additional ingredients, such as proteins (e.g., hydrolyzed vegetable protein, hydrolyzed wheat protein, hydrolyzed milk protein, hydrolyzed silk and hydrolyzed collagen), vitamins (e.g., panthenol, biotin, vitamin A and D palmitate), moisturizers/humectants (e.g., glycerin, propylene glycol, sodium pyroglutamic acid (also known as PCA), amino acid-based surfactants, and HLA), emollients (e.g., esters, isopropyl myristate, decyl oleate, C12-15 alkyl benzoate), botanicals (e.g., chamomile, aloe, rosemary), as well as preservatives, dyes, pH adjusters and chelating agents. For instance, in some embodiments, the personal care composition includes milk protein and/or hydrolyzed milk protein in an amount or range described above with respect to the additional ingredients. Additional examples of vitamins that may be included in some personal care compositions include tocopherol, retinol, and ascorbic acid.

Non-limiting examples of fragrances and perfumes include odor compounds selected from: 7-acetyl-1,2,3,4,5,6,7,8-octahydro-1,1,6,7-tetramethylnaphthalene, α-ionone, β-ionone, γ-ionone α-isomethylionone, methylcedrylone, methyl dihydrojasmonate, methyl 1,6,10-trimethyl-2,5,9-cyclododecatrien-1-yl ketone, 7-acetyl-1,1,3,4,4,6-hexamethyltetralin, 4-acetyl-6-tert-butyl-1,1-dimethylindane, hydroxyphenylbutanone, benzophenone, methyl β-naphthyl ketone, 6-acetyl-1,1,2,3,3,5-hexamethylindane, 5-acetyl-3-isopropyl-1,1,2,6-tetramethylindane, 1-dodecanal, 4-(4-hydroxy-4-methylpentyl)-3-cyclohexene-1-carboxaldehyde, 7-hydroxy-3,7-dimethyloctanal, 10-undecen-1-al, isohexenylcyclohexylcarboxaldehyde, formyltricyclodecane, condensation products of hydroxycitronellal and methyl anthranilate, condensation products of hydroxycitronellal and indole, condensation products of phenylacetaldehyde and indole, 2-methyl-3-(para-tert-butylphenyl) propionaldehyde, ethylvanillin, heliotropin, hexylcinnamaldehyde, amylcinnamaldehyde, 2-methyl-2-(isopropylphenyl) propionaldehyde, coumarin, γ-decalactone, cyclopentadecanolide, 16-hydroxy-9-hexadecenoic acid lactone, 1,3,4,6,7,8-hexahydro-4,6,6,7,8,8-hexamethylcyclopenta-γ-2-benzopyran, β-naphthol methyl ether, ambroxane, dodecahydro-3a,6,6,9a-tetramethylnaphtho [2,1b]furan, cedrol, 5-(2,2,3-trimethylcyclopent-3-enyl)-3-methylpentan-2-ol,2-ethyl-4-(2,2,3-trimethyl-3-cyclopenten-1-yl)-2-buten-1-ol, caryophyllene alcohol, tricyclodecenyl propionate, tricyclodecenyl acetate, benzyl salicylate, cedryl acetate, and tert-butylcyclohexyl acetate.

Other fragrances may include odor compounds selected from essential oils, resinoids and resins from a large number of sources, such as, for example, Peru balsam, olibanum resinoid, styrax, labdanum resin, nutmeg, cassia oil, benzoin resin, coriander, and lavandin.

Further suitable fragrances include odor compounds selected from phenylethyl alcohol, terpineol, linalool, linalyl acetate, geraniol, nerol, 2-(1,1-dimethylethyl) cyclo-hexanol acetate, benzyl acetate, and eugenol. The fragrances or perfumes can be used as single substances or in a mixture with one another.

The personal care composition may further comprise one or more colorants. The colorants may be a pigment, a dye, or mixtures thereof. Non-limiting examples of pigments include titanium dioxide, zinc oxide, kaolin, mica, and combinations thereof. Non-limiting examples of dyes include food dyes suitable for food, drug and cosmetic applications, and mixtures thereof. Some color agents (colorants) are known as FD&C dyes.

The colorants may be present in an amount ranging from about 0.0001 wt. % to about 0.4 wt. %, including all percentages and subranges therebetween, based on the total weight of the personal care composition. In some embodiments, the colorants may be present in an amount ranging from about 0.0001 to about 4 wt. %, including all percentages and subranges therebetween, based on the total weight of the personal care composition.

In certain embodiments, in order to prevent ingredients from separating, emulsifiers may be added. In certain embodiments, additional ingredients may include conditioning agents that moisturize the skin during and after use of the personal care composition.

In other embodiments, the personal care composition may include any of following materials in any desired amount to achieve a desired effect in the composition (amounts that can be used in some embodiments are provided): one or more alkaline salts, for example, sodium chloride, sodium sulfate, sodium carbonate, sodium bicarbonate and/or their equivalents (0 to 5 wt. %); sequestrants, for example, tetrasodium EDTA, and/or their equivalents (0 to 2 wt. %); biocides, for example, Triclosan (2,4,4′-trichloro-2′-hydroxydiphenyl ether), DMDM hydantoin, formaldehyde and/or imidazolidinyl urea, and/or their equivalents (0 to 2 wt. %); organic acids, for example, citric acid and/or formic acid and/or their equivalents (0 to 2 wt. %); viscosity modifiers (0 to 2 wt. %); fragrances and/or perfumes (0 to 5 wt. %); preservatives, for example, phenoxyethanol, formaldehyde solution, parabens, pentanediol or sorbic acid (0 to 2 wt. %); pearlizing agents, for example, glycol distearic esters, such as ethylene glycol distearate, but also fatty acid monoglycol esters (0 to 3 wt. %); stabilizers, for example, metal salts of fatty acids, such as e.g. magnesium stearate, aluminum stearate and/or zinc stearate (0 to 2 wt. %); and dyes and pigments that are approved and suitable for cosmetic purposes.

The personal care compositions may be aqueous and, e.g., include water in an amount from about 25 to about 96 wt. %, based on the total weight of the personal care composition. For example, the water may be present in the personal care composition in an amount from about 30 to about 96 wt. %, about 40 to about 96 wt. %, about 50 to about 96 wt. %, about 60 to about 96 wt. %, about 70 to about 96 wt. %, about 80 to about 96 wt. %, about 85 to about 96 wt. %; from about 25 to about 90 wt. %, about 40 to about 90 wt. %, about 50 to about 90 wt. %, about 60 to about 90 wt. %, about 70 to about 90 wt. %, about 80 to about 90 wt. %, about 85 to about 90 wt. %; from about 25 to about 85 wt. %, about 40 to about 85 wt. %, about 50 to about 85 wt. %, about 60 to about 85 wt. %, about 70 to about 85 wt. %; from about 25 to about 80 wt. %, about 40 to about 80 wt. %, about 50 to about 80 wt. %, about 60 to about 80 wt. %, about 70 to about 80 wt. %; from about 25 to about 70 wt. %, about 40 to about 70 wt. %, about 50 to about 70 wt. %, about 60 to about 70 wt. %; from about 25 to about 60 wt. %, about 40 to about 60 wt. %, about 50 to about 60 wt. %; from about 25 to about 50 wt. %, about 40 to about 50 wt. %, about 50 to about 50 wt. %, about 60 to about 50 wt. %, or any range or subrange thereof, based on the total weight of the personal care composition.

In some embodiments, the amount of water in the personal care composition may be from about 1 to about 35 wt. %, based on the total weight of the personal care composition. For example, the personal care composition may include water in an amount from about 1 to about 20 wt. %, about 1 to about 10 wt. %, about 1 to about 8 wt. %, about 1 to about 6 wt. %, about 1 to about 4 wt. %, about 1 to about 3 wt. %; from about 3 to about 20 wt. %, about 3 to about 10 wt. %, about 3 to about 8 wt. %, about 3 to about 6 wt. %; from about 5 to about 30 wt. %, about 5 to about 25 wt. %, about 5 to about 21 wt. %, about 5 to about 17 wt. %, about 5 to about 14 wt. %, about 5 to about 11 wt. %; from about 10 to about 35 wt. %, about 10 to about 30 wt. %, about 10 to about 25 wt. %, about 10 to about 21 wt. %, about 10 to about 17 wt. %, about 10 to about 14 wt. %; from about 15 to about 35 wt. %, about 15 to about 30 wt. %, about 15 to about 25 wt. %, about 15 to about 21 wt. %, about 15 to about 19 wt. %; from about 18 to about 35 wt. %, about 18 to about 30 wt. %, about 18 to about 25 wt. %, about 18 to about 21 wt. %; from about 21 to about 35 wt. %, about 21 to about 30 wt. %, about 21 to about 25 wt. %; from about 24 to about 35 wt. %, about 24 to about 30 wt. %; from about 27 to about 35 wt. %, about 27 to about 30 wt. %, or any range or subrange thereof, based on the total weight of the personal care composition.

The personal care composition may, in some instances, comprise tallow and/or soap chips. The amount of tallow and/or soap chips in the personal care composition may be from about 40 to about 85 wt. %, based on the total weight of the personal care composition. For instance, the personal care composition may include a total amount of tallow and/or soap in an amount from about 40 to about 85 wt. %, about 40 to about 80 wt. %, about 40 to about 75 wt. %, about 40 to about 70 wt. %, about 40 to about 65 wt. %, about 40 to about 60 wt. %, about 40 to about 55 wt. %; from about 50 to about 85 wt. %, about 50 to about 80 wt. %, about 50 to about 75 wt. %, about 50 to about 70 wt. %, about 50 to about 65 wt. %, about 50 to about 60 wt. %; from about 55 to about 85 wt. %, about 55 to about 80 wt. %, about 55 to about 75 wt. %, about 55 to about 70 wt. %, about 55 to about 65 wt. %; from about 60 to about 85 wt. %, about 60 to about 80 wt. %, about 60 to about 75 wt. %, about 60 to about 70 wt. %; from about 65 to about 85 wt. %, about 65 to about 80 wt. %, about 65 to about 75 wt. %, about 65 to about 70 wt. %; from about 70 to about 85 wt. %, about 70 to about 80 wt. %, about 75 to about 85 wt. %, including any range or subrange thereof, based on the total weight of the personal care composition. The soap chips may be coco soap chips or the like.

The personal care compositions may also include caustic soda, e.g., in an amount from about 1 to about 25 wt. %, based on the total weight of the personal care composition. In some embodiments, the amount of soap chips in the personal care composition is from about 1 to about 20 wt. %, about 1 to about 10 wt. %, about 1 to about 8 wt. %, about 1 to about 6 wt. %, about 1 to about 4 wt. %, about 1 to about 3 wt. %; from about 3 to about 20 wt. %, about 3 to about 10 wt. %, about 3 to about 8 wt. %, about 3 to about 6 wt. %; from about 5 to about 25 wt. %, about 5 to about 21 wt. %, about 5 to about 17 wt. %, about 5 to about 14 wt. %, about 5 to about 11 wt. %; from about 10 to about 25 wt. %, about 10 to about 21 wt. %, about 10 to about 17 wt. %, about 10 to about 14 wt. %; from about 15 to about 25 wt. %, about 15 to about 21 wt. %, about 15 to about 19 wt. %; from about 18 to about 25 wt. %, about 18 to about 21 wt. %; from about 21 to about 25 wt. %, or any range or subrange thereof, based on the total weight of the personal care composition.

The personal care composition may, in some cases, include one or more fatty acid(s). The one or more fatty acid may be present in an amount from about 1 to about 15 wt. %, based on the total weight of the personal care composition. In some instance, the one or more fatty acid(s) may be present in an amount from about 1 to about 14 wt. %, about 1 to about 13 wt. %, about 1 to about 11 wt. %, about 1 to about 9 wt. %, about 1 to about 7 wt. %, about 1 to about 5 wt. %, about 1 to about 3 wt. %; from about 2 to about 15 wt. %, about 2 to about 13 wt. %, about 2 to about 11 wt. %, about 2 to about 9 wt. %, about 2 to about 7 wt. %, about 2 to about 5 wt. %; from about 4 to about 15 wt. %, about 4 to about 13 wt. %, about 4 to about 11 wt. %, about 4 to about 9 wt. %, about 4 to about 7 wt. %; from about 6 to about 15 wt. %, about 6 to about 13 wt. %, about 6 to about 11 wt. %, about 6 to about 9 wt. %; from about 8 to about 15 wt. %, about 8 to about 13 wt. %, about 8 to about 11 wt. %; from about 10 to about 15 wt. %, about 10 to about 13 wt. %, or any range or subrange thereof, based on the personal care composition.

In some instances, the fatty acids may be chosen from fatty acid derivatives, esters of fatty acids, hydroxyl-substituted fatty acids, and alkoxylated fatty acids. The fatty acids may be straight or branched chain acids and/or may be saturated or unsaturated. Non-limiting examples of fatty acids include diacids, triacids, and other multiple acids as well as salts of these fatty acids. For example, the fatty acid may optionally include or be chosen from lauric acid, palmitic acid, stearic acid, behenic acid, arichidonic acid, oleic acid, isostearic acid, sebacic acid, and a mixture thereof. In some cases, the fatty acids are selected from the group consisting of palmitic acid, stearic acid, and a mixture thereof.

Non-limiting examples of polyglycerol esters of fatty acids include those of the following formula:

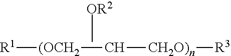

wherein the average value of n is about 3 and R¹, R² and R³ each may independently be a fatty acid moiety or hydrogen, provided that at least one of R¹, R², and R³ is a fatty acid moiety. For instance, R¹, R² and R³ may be saturated or unsaturated, straight or branched, and have a length of C1-C40, C1-C30, C1-C2s, or C1-C20, C1-C16, or C1-C10.

The fatty acid derivatives are defined herein to include fatty acid esters of the fatty alcohols as defined above, fatty acid esters of the fatty alcohol derivatives as defined above when such fatty alcohol derivatives have an esterifiable hydroxyl group, fatty acid esters of alcohols other than the fatty alcohols and the fatty alcohol derivatives described above, hydroxy-substituted fatty acids, and a mixture thereof. Non-limiting examples of fatty acid derivatives include ricinoleic acid, glycerol monostearate, 12-hydroxy stearic acid, ethyl stearate, cetyl stearate, cetyl palmitate, polyoxyethylene cetyl ether stearate, polyoxyethylene stearyl ether stearate, polyoxyethylene lauryl ether stearate, ethyleneglycol monostearate, polyoxyethylene monostearate, polyoxyethylene distearate, propyleneglycol monostearate, propyleneglycol distearate, trimethylolpropane distearate, sorbitan stearate, polyglyceryl stearate, dimethyl sebacate, PEG-15 cocoate, PPG-15 stearate, glyceryl monostearate, glyceryl distearate, glyceryl tristearate, PEG-8 laurate, PPG-2 isostearate, PPG-9 laurate, and a mixture thereof. Preferred for use herein are glycerol monostearate, 12-hydroxy stearic acid, and a mixture thereof.

Aspects of the invention are directed to methods for preserving pigment associated with a skin tattoo, improving the health skin, and/or improving the appearance of skin by applying a personal care composition disclosed herein. In some embodiments, the methods include applying one or more (e.g., two, three, or four) personal care compositions. The methods may employ the personal care compositions disclosed herein.

The methods typically include applying an effective amount of the personal care composition to a section of skin. The amount of the personal care composition applied to the skin may be about 0.1 to about 5 grams/inch² of skin. In some cases, the amount of personal care composition applied to the skin is about 0.1 to about 3 grams/inch², about 0.1 to about 2 grams/inch², about 0.1 to about 1 grams/inch²; about 0.5 to about 3 grams/inch², about 0.5 to about 2 grams/inch², about 0.5 to about 1 grams/inch²; about 1 to about 3 grams/inch², about 1 to about 2 grams/inch²; about 1.5 to about 3 grams/inch², about 1.5 to about 2 grams/inch², including any ranges or subranges therebetween. The personal composition may be spread on the skin by hand, or it may be done by an instrument such as a glove or a piece of cloth.

The personal composition may be allowed to remain on the skin for a period of time sufficient to clean the section of skin—for example, for about 1 second to about 3 minute, about 1 second to about 1 minute, about 1 to about 45 seconds, about 1 to about 30 seconds, or about 1 to about 15 seconds. As is common, the skin may be wetted or rinsed with water prior to the application of the personal care composition. Having the skin wetted before applying the personal care composition may be beneficial for enhancing the effects of certain personal care compositions according to aspects of the invention.

The method may, in some cases, include a step of drying the section of skin before applying the personal care composition the section of skin. The skin may be dried by towel, hair drier, or other means, e.g., depending on the desired degree of dryness before the application of the lotion composition.

### EXAMPLES

### Example 1

Six personal care compositions (Compositions A-F) were prepared in the form of a rinse off body wash in accordance with the formulas shown in Table 1.

TABLE 1

| | Comp. A | Comp. B | Comp. C | Comp. D | Comp. E | Comp. F |
|---|---|---|---|---|---|---|
| Ingredient Name | (wt. %) | (wt. %) | (wt. %) | (wt. %) | (wt. %) | (wt. %) |
| C₁₂₋₁₄ Alcohol EO 2:1 Na | 8.3 | 8.3 | 8.3 | 8.3 | 8.3 | 8.3 |
| Sulfate | | | | | | |
| Cocoamidopropyl Betaine | 8.5 | 8.5 | 8.5 | 8.5 | 8.5 | 8.5 |
| Lactic Acid | 1.8 | 1.8 | 1.8 | 1.8 | 1.8 | 1.8 |
| Polyquaternium-7 | 0.6 | 0.6 | 0.6 | 0.6 | 0.6 | 0.6 |
| Cocomonoethanolamide | 0.5 | 0.5 | 0.5 | 0.5 | 0.5 | 0.5 |
| NA₂O Caustic Soda | 0.2 | 0.2 | 0.2 | 0.2 | 0.2 | 0.2 |
| Pearlescent Base | 0.5 | 0.5 | 0.5 | 0.5 | 0.5 | 0.5 |
| EQUILIBRIO 2020 | 1.1 | 1.1 | 1.1 | 1.1 | 1.1 | 1.1 |
| ALKESTE 150 D VG | 0.25 | 0.25 | 0.25 | 0.25 | 0.25 | 0.25 |
| (PEG 6000) | | | | | | |
| EDTA | 0.22 | 0.22 | 0.22 | 0.22 | 0.22 | 0.22 |
| Flax Seed Oil | 0.2 | 0.2 | 0.2 | 0.2 | 0.2 | 0.2 |
| PEG-18 Castor Oil | 0.2 | 0.2 | 0.2 | 0.2 | 0.2 | 0.2 |
| Dioleate | | | | | | |
| Sodium Benzoate | 0.15 | 0.15 | 0.15 | 0.15 | 0.15 | 0.15 |
| Glycerin | 0.0001-0.1 | 0.0001-0.1 | 0.0001-0.1 | 0.0001-0.1 | 0.0001-0.1 | 0.0001-0.1 |
| Vitamin E, N, F (ALPHA) | 0.01 | 0.01 | 0.1 | | 0.1 | 0.1 |
| Bisabolol | | | | | 0.25 | 0.125 |
| Niacinamide USP | 0.2 | | | | | |
| Tocopherol acetate | 0.1 | 0.1 | | 0.1 | | |
| Sodium Ascorbil | | 0.02 | | 0.01 | | |
| Phosphate | | | | | | |
| Water | Q.S. | Q.S. | Q.S. | Q.S. | Q.S. | Q.S. |
| | to 100 | to 100 | to 100 | to 100 | to 100 | to 100 |

### Example 2

Composition A from Example 1 was evaluated to assess whether the niacinamide would be rinsed off during use. A sample of Composition A was applied to 2 inch by 2 inch pieces of porcine back skin (provided by Animal Technologies) with the subcutaneous fat removed. For the rinse off procedure, the porcine back skin was rinsed with water having a temperature of 100° F. at a flow rate of 6L/min. Specifically, the porcine back skin was first wetted for five seconds. Then, 50 uL of Composition A was applied to the porcine back skin and rubbed on for 10 seconds. The porcine back skin was then allowed to rest for 90 seconds and then rinsed off with water for 15 seconds. After letting the skin air dry for 5 minutes, a glass cup (3 cm. diameter) was applied to the surface of the skin. 3 mL of ethanol was applied within the cup, rubbed for 30 seconds with a glass rod, and then extracted into a labeled scintillation vial. This was repeated once more, for a total of 6 mL of extracted ethanol. The samples were left to air dry. Afterwards, samples were resolubilized in 1 mL of a 90/10 v/v mixture of acetonitrile/water and filtered into an HPLC vial. The samples were then run on the HPLC for niacinamide quantification.

A HPLC, namely a Agilent 1100 Series HPLC with a UV-Vis detector, was used for niacinamide quantification. A normal phase, Agilent Poroshell 120 HILIC-Z column (150 mm×4.6 mm) was employed. The mobile phase consisted of 90/10 acetonitrile/water, which was run at a flow rate of 1 mL/min. 20 uL of solution was injected. Niacinamide could be detected at 260 nm with a retention time of about 2.2 minutes.

The high water solubility of niacinamide suggests that most of it will be washed off by water during the rinsing process. Therefore, to maximize the possibility of detecting niacinamide, samples were evaporated to dryness and then reconstituted in a minimal amount of mobile phase solvent, to increase the concentration of niacinamide present.

The HPLC analysis revealed a significant peak at the retention time for niacinamide, indicating that niacinamide unexpectedly remained after rinsing. Specifically, the level of deposited niacinamide was calculated to be 0.065 +/−0.030 ug/cm².

### Example 3

Compositions A-F were evaluated to assess if the respective compositions reduce degradation of pigments associated with tattoos. Specifically, each of Compositions A-F were evaluated using a malondialdehyde (MDA) evaluation to assess the antioxidant effects of the respective compositions. Equal amount of test formulation or placebo is applied to skin and rubbed to mimic washing. An untreated piece of skin also undergoes rubbing, but no product application, and serves as control. The skin samples are all rinsed, dried and exposed to oxidative stress. Lipids are then extracted using the cup scrub method, and the amount of lipid peroxidation is evaluated using an Malondialdehyde (MDA) Lipid Peroxidation Kit (Millipore-Sigma).

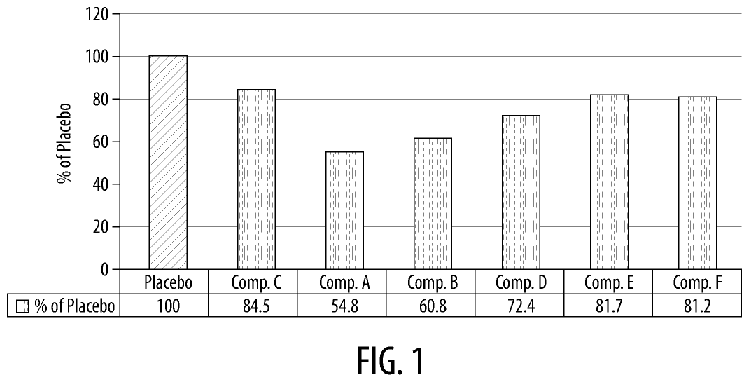

A summary of the results are shown in FIG. 1. In FIG. 1, a lower value indicates a higher antioxidant effect.

### Example 4

Two personal care compositions (Compositions G and H) were prepared in the form of a rinse off body soap bar in accordance with the formulas shown in Table 2.

TABLE 2

| | Comp. G | Comp. H |
|---|---|---|
| | (wt. %) | (wt. %) |
| Crude Tallow | 65.6 | |
| Sodium Soap | | 70.5 |
| NA₂O Caustic Soda | 7.7 | |
| Sodium Dodecyl Benzene | 1.9 | 1.9 |
| Sulfonate | | |
| Lauric Acid and/or | 5.2 | 1.5 |
| Free Fatty Acid | | |
| Fatty Acid Ester | | 0.05-0.1 |
| Cocomonoethanolamide | 1.9 | |
| Cocoamidopropyl Betaine | 1.6 | 0.5 |
| Cocamide Mea | | 1.9 |
| Citric Acid or Sodium Citrate | 0.5 | 0.7 |
| Hydrolyzed Milk Protein | 1 | 0.2 |
| Miscellaneous (Fragrance, | about 2 | about 1 |
| Pigment, titanium dioxide, Iron | | |
| Oxide, and/or Sodium Benzoate) | | |
| Sodium Chloride | 0.6 | 0.9 |
| Flax Seed Oil Virgin | 0.5 | |
| Niacinamide | 0.2 | 0.2 |
| d-Gamma Tocopherol | 0.03 | 0.03 |
| Tetrasodium EDTA - | 0.05-0.1 | 0.01-0.05 |
| Etidronic Acid | 0.01-0.1 | 0.01-0.1 |
| Glycerin | | 6.9 |
| Linum Usitatissimum (Linseed) | | 0.5 |
| Seed Oil | | |
| Sodium Carbonate | | 0.01-0.1 |
| Ethanolamine | | 0.01-0.1 |
| Water | Q.S. to 100 | Q.S. to 100 |

### Example 5

Composition G from Example 4 was evaluated to assess whether the niacinamide would be rinsed off during use. Compositions G was evaluated in accordance with the procedure described in Example 2. As noted above, the high water solubility of niacinamide suggests that most of it will be washed off by water during the rinsing process. The HPLC analysis, however. revealed a significant peak at the retention time for niacinamide, indicating that niacinamide unexpectedly remained after rinsing. Specifically, the level of deposited niacinamide was calculated to be 0.0086 +/−0.039 ug/cm².

What is claimed is:

1. A personal care composition comprising:
a cosmetically acceptable carrier; and
a color protection component comprising:
from about 0.01 to about 5 wt. % of one or more vitamin E, and
from about 0.01 to about 7 wt. % of one or more vitamin B comprising niacinamide, wherein all weight percentages are based on the total weight of the personal care composition.

2. A personal care composition comprising:
a cosmetically acceptable carrier; and
a color protection component comprising:
from about 0.01 to about 5 wt. % of one or more vitamin E, and
from about 0.01 to about 5 wt. % of one or more vitamin C comprising ascorbyl phosphate,
wherein all weight percentages are based on the total weight of the personal care composition.

3. The personal care composition according to claim 1, wherein the one or more vitamin E comprises vitamin E acetate, vitamin E succinate, alpha-tocopherol, beta-tocopherol, gamma-tocopherol, delta-tocopherol, alpha-tocotrienol, beta-tocotrienol, gamma-tocotrienol, delta-tocotrienol, tocopherol acetate, tocopherol phosphate, or any combination thereof.

4. The personal care composition according to claim 1, wherein the one or more vitamin E comprises alpha-tocopherol and vitamin E acetate.

5. The personal care composition according to claim 1, wherein the personal care composition has a weight ratio of the total amount of vitamin E to niacinamide of from about 2:1 to about 1:6.

6. The personal care composition according to claim 5, wherein the weight ratio of the total amount of vitamin E to niacinamide is from about 1:1 to about 1:3.

7. The personal care composition according to claim 2, wherein the personal care composition has a weight ratio of the total amount of vitamin E to ascorbyl phosphate of from about 14:1 to about 1:2.

8. The personal care composition according to claim 7, wherein the has a weight ratio of the total amount of vitamin E to ascorbyl phosphate is from about 7:1 to about 3:1.

9. The personal care composition according to claim 1, further comprising from about 0.5 to about 9 wt. % of lactic acid and/or a salt thereof.

10. The personal care composition according to claim 1, further comprising from about 0.5 to about 9 wt. % of an oil, the oil comprising flax seed oil, castor oil, or a combination thereof.

11. The personal care composition according to claim 1, further comprising from about 5 to about 45 wt. % of one or more surfactants.

12. The personal care composition according to claim 11, wherein the one or more surfactant comprises cocamidopropyl betaine.

13. The personal care composition according to claim 1, wherein the color protection component further comprises from about 0.01 to about 10 wt. % of one or more vitamin C, wherein the one or more vitamin C is selected from ascorbyl phosphate, ascorbyl glucoside, and a combination of two or more thereof.

14. The personal care composition according to claim 2, wherein the color protection component further comprises from about 0.01 to about 10 wt. % of one or more vitamin B.

15. The personal care composition according to claim 14, wherein the one or more vitamin B comprises niacinamide.

16. The personal care composition according to claim 1, wherein the personal care composition reduces the amount of color change by at least 25 wt. %.

17. The personal care composition according to claim 16, wherein the color change is assessed by a malondialdehyde evaluation relative to a placebo, wherein the placebo is free of vitamin B, vitamin C, and vitamin E.

18. The personal care composition according to claim 1, wherein the personal care composition reduces the amount of color change by at least 25 wt. % as assessed by the difference between the amount of a light having a wavelength of 428 nm adsorbed by CI 19140 sample containing personal care composition before the irradiation with UVA+UVB and after irradiation with UVA+UVB, as compared to the difference between the amount of a light having a wavelength of 428 nm adsorbed by a CI 19140 sample containing placebo personal care composition before irradiation with UVA+UVB and after irradiation with UVA+UVB, wherein the placebo has a similar formulation as the personal care composition, but does not include the active ingredients (e.g., free of vitamin B, vitamin C, and vitamin E).

19. The personal care composition according to claim 1, wherein the personal care composition is in the form of a rinse off composition.

20. The personal care composition according to claim 1, wherein the personal care composition is in the form of a leave on composition.
